(19) United States
(12) Patent Application Publication
(10) Pub. No.: US 2010/0145319 A1
(43) Pub. Date: Jun. 10, 2010

(54) COAGULATION SYSTEM

(75) Inventors: Diego Zimare, Pausa (DE); Manfred Dick, Gefell (DE)

Correspondence Address: Darby & Darby, P.O. Box 770, Church Street Station, New York, NY, 10008-0770, US

(73) Assignee: CARL ZEISS MEDITEC AG, JENA (DE)

(21) Appl. No.: 12/525,994

(22) PCT Filed: Feb. 1, 2008
(86) PCT No.: PCT/EP08/00834

§ 371 Date: Feb. 23, 2010

(30) Foreign Application Priority Data
Feb. 5, 2007 (DE) 102007005699.2

(51) Int. Cl. A61F 9/008; A61B 18/20

(52) U.S. Cl. 606/4; 606/12

(57) ABSTRACT

A coagulation system for the coagulation of organic tissue includes a laser configured to emit a working beam; an interrupter configured to at least partially interrupt the working beam; a controller configured to activate the interrupter; and a detector configured to detect a signal corresponding to a degree of coagulation or alteration of the tissue and to transmit a detection of the signal to the controller, the detector including a dimension meter.

This is a U.S. National Phase Application under 35 U.S.C. §171 of PCT/EP2008/000834, filed on Feb. 1, 2008, which claims priority to German Application No. DE 102007005699.2, filed on Feb. 5, 2007. The International Application was published in German on Aug. 14, 2008 as WO 2008/095659 under PCT article 21(2).

The invention relates to a coagulation system for the coagulation of organic tissues.

### BACKGROUND

Photocoagulation was first used at the end of the 1940s by means of the focused light of an axial high-pressure lamp to treat various diseases of the retina—for example diabetic retinopathy. By absorbing the laser beam particularly in the pigment epithelium, a layer located in the retina and bearing a dark pigment, in particular melanin, the retina is heated and coagulated. The metabolism is thereby focused on the still healthy areas of the retina. In addition, biochemical cofactors are stimulated. The course of the disease is thus clearly slowed or stopped.

A process for operating a photocoagulator for biological tissue is described in DE 30 24 169. A device for thermally altering biological tissue is described in DE 39 36 716.

### SUMMARY OF THE INVENTION

However, a disadvantage with the devices described in the two published documents is that, during their use, tissue which is worth preserving, in particular the photoreceptor layer located in the direction of the beam in front of the retinal pigment epithelium, is destroyed.

An aspect of the present invention is to provide a coagulation system for the coagulation of organic tissues which minimizes the destruction of tissue worth preserving.

The present invention provides a coagulation system for the coagulation of organic tissues, particularly of the retina, which comprises a laser, a detector, a controller and an interrupter, wherein the laser is set up to emit a working beam, the detector has a dimension meter and is set up to detect a signal and to transmit the detection of a signal to the controller, the controller is set up to activate an interrupter, the interrupter is set up to interrupt the emission of radiation with at least one wavelength of the working beam of the laser, and the signal corresponds to a degree of coagulation or alteration of the tissue. Preferably the controller is set up to trip the interrupter when the detector signals exceed predefined limit values.

Argon lasers, diode lasers, diode-pumped solid-state lasers, diode-pumped semiconductor lasers, frequency-doubled Nd:YAG lasers, etc. are preferably used. The lasers can be used pulsed or as CW lasers. In addition further light sources are also conceivable, such as for example focused light of a xenon lamp, of light-emitting diodes (LEDs), superluminescent diodes (SLDs), etc. Laser systems, particularly preferably multi-wavelength systems, which are set up to emit waves with green, yellow, red and infrared wavelengths are preferably used. Wavelengths used in particular for coagulation lie in the green wavelength range (514 nm, 532 nm), where the comparatively highest absorption of the photopigment melanin is found, or in the yellow spectral range (561-580 nm), where the absorption of the blood pigment haemoglobin is at its greatest. For coagulations of high penetration depth, red wavelengths (630-690 nm) or infrared wavelengths, such as e.g. 810 nm, are used.

Any equipment which can detect the presence of radiation is suitable as the detector. The detector is preferably suitable for determining radiance and/or direction of radiation and/or a wavelength of the radiation. The detector is preferably a smart sensor which has a microprocessor. A photocell, a photodiode or a photomultiplier, particularly preferably a semiconductor detector, preferably made of silicon or germanium, such as for example a charge-coupled device, particularly preferably a bolometer or pyrometer, is preferably used as the detector. The detectors are preferably arranged in a detection system. The detection system is preferably an interferometer.

Any equipment which can control a device depending on an input quantity is suitable as the controller. The control means preferably has both at least one input interface and at least one output interface. Preferably the control means can be programmed. A hard-wired programmed control means is preferably used, particularly preferably a stored-programmed control means. The control means preferably has a processor architecture.

Any equipment which is set up to wholly or partly interrupt the working beam of a laser is suitable as the interrupter. Preferably, a device which switches the laser off is used as the interrupter. Particularly preferably, a diaphragm which is suitable for limiting or interrupting the working beam of a laser is used. For this, the diaphragm is preferably positioned in an area through which the laser beam passes. Particularly preferably, a filter is used as the interrupter. The filter can preferably be guided into the working beam in order to interrupt it. The filter in particular interrupts only some of the working beam, preferably the filter interrupts only waves of the working beam which have a specific length, particularly preferably the filter interrupts the whole of the working beam.

The interrupter is preferably set up to interrupt the emission of individual waves with specific wavelengths of a laser or laser system which preferably emits waves with wavelengths in the visible range, particularly preferably waves with wavelengths in the infrared range. Preferably, waves with wavelengths in the visible range, particularly preferably waves with wavelengths in the infrared range, are interrupted. Particularly preferably, the interrupter is also set up to interrupt waves independently of a detected coagulation. Thus coagulation times pre-selected for individual patients can also be tripped in series using the interrupter.

Preferably, the intensity of waves with specific wavelengths by which the organic tissue is irradiated is thereby altered during the irradiation. Particularly preferably, the emission of waves which have a specific wavelength or one of several specific wavelengths is interrupted for the whole duration of the irradiation. Thus it is made possible to stop the irradiation depending on the detection signal. Moreover, it is possible to alter the irradiation depending on time. In particular the penetration depth of the radiation is thereby controlled. In this way it is possible to irradiate the tissue over the duration of the irradiation in a targeted manner in each case with the wavelengths which are best suited to bring about a coagulation and to avoid damage to the surrounding tissue. Preferably, the emission of radiation with wavelengths in the infrared range is interrupted in order to limit penetration deep into the tissue. Infrared radiation has wavelengths of 780 nm to 1 mm.

The working beam is preferably parallel, particularly preferably bundled. The working beam is preferably non-polarized, particularly preferably polarized. A polarized working beam is elliptical, preferably circular, particularly preferably linear. The working beam has one wavelength, preferably several different wavelengths. These wavelengths preferably lie in the visible and/or infrared range.

Any equipment which is set up to measure dimensions or corresponding effects of the radiation action can be used as the dimension meter. Preferably, the dimension meter is set up to assess the effect of the applied radiation. A measuring microscope is preferably used, particularly preferably an interferometric device, a confocal device or an optical coherence tomography device or OCT device. The OCT device preferably operates according to the “time-domain principle”, particularly preferably according to the “spectral domain (Fourier) principle”. An interferometric device is any device by which an interference pattern can be generated. A Twyman-Green interferometer, preferably a Mach-Zehnder interferometer, particularly preferably a Fabry-Pérot interferometer or a Michelson interferometer is used as the interferometric device. A device which has a lens for focusing light into the sample is preferably used as the confocal device. Particularly preferably, the confocal device has a light source, a beam splitter and two pinhole diaphragms. Preferably, light from the light source is conducted through the pinhole diaphragm, the beam splitter and the lens into the sample, and from there conducted through the lens again onto the beam splitter and the pinhole diaphragm. A device which has a light source, a beam splitter, a measuring arm, a reference arm with a mirror and a detector is preferably used as the OCT device. Preferably, beams of the light source are conducted through the beam splitter to the mirror, the measuring arm and the detector. The running time of the light on the reference arm and the measuring arm is preferably compared. Particularly preferably, the interference of the individual spectral components is recorded. Light sources which provide a short coherence length are preferably used as the light source or radiation source. Broadband superluminescent diodes, fs-lasers or swept-source systems are preferably used. An OCT signal is preferably based on scattering, particularly preferably absorption.

The signal is in particular reflected and/or scattered, preferably fluorescent and particularly preferably thermally or audibly generated. The signal is preferably generated by the exceeding of a limit value for specific properties of the radiation reflected by the organic tissue. The signal is preferably the exceeding of a specific brightness value or of a value for the scattering, particularly preferably the exceeding of a specific temperature value, an acoustic pressure gradient or a wavelength.

Waves here are spreading vibrations. Preferably, they are shock waves, particularly preferably periodic waves.

The wavelength here is the smallest distance between two points of the same phase of a wave. The two points of the same phase display the same deflection and the same direction of movement in the time sequence.

The degree of change of the tissue preferably corresponds to a change in the spectral properties (change of colour) or a change in the mechanical properties, such as for example hardness or elasticity, particularly preferably to a change in the temperature of the tissue or its acoustic effects.

The laser preferably has a clock generator to pulse the working beam over a period of time which lies between 10 ms and 10,000 ms. Particularly preferably, clock generators or rapid-action switches are used for emission durations in range of 0.1 μs to 10 ms. The electrical switching of the pump energy is preferably used as the clock generator for the pulsing or rapid switching of the laser emission. Particularly preferably, electromechanical, acousto-optic (Bragg cells) or electro-optic switches (Pockels cells) are used to interrupt the beam path inside or outside the resonator.

By pulsed output is meant here that a specific performance is delivered at recurring identical intervals of time. The period of time in which the performance is delivered is also preferably the same in each case.

In traditional laser coagulation, pre-set irradiation times in the range of preferably 10 ms to 10,000 ms, particularly preferably approx. 100 ms are preferably set. The pulse is preferably activated by the doctor to emit in single-shot operation once he has found the position on the fundus of the eye using the target beam. In the more gentle selective retina therapy (SRT), pulse lengths are preferably set in the range of 0.1 μs to approx. 10 ms, particularly preferably 1-5 μs. The irradiation times are also pre-set in fixed manner. The pulse is triggered to emit in single-shot operation by the doctor once he has found the position on the fundus of the eye likewise using the target beam.

While in the case of traditional laser coagulation the doctor can also easily recognize the fixed coagulation centres visually during follow-up examinations, in the case of a gentle SRT treatment this is still possible only in an angiographic image. In the colour picture of a normal slit lamp diagnosis with a contact glass or of a fundus camera, a sufficient visibility of previous treatment areas is not recognizable.

The laser preferably has a pulse generator to pulse the working beam with a pulse duration of between 2 and 10 μs, preferably between 3 and 7 μs, particularly preferably 5 μs.

A damper is preferably fitted into the optical resonator as the pulse generator. Particularly preferably, an acousto-optical modulator or a saturable absorber is used as the pulse generator.

When switched on, the damper fitted into the resonator prevents the reflection of light which is emitted. No radiation is thereby delivered.

A selector which can switch very quickly (<10 ns) between blocking and letting through is preferably used as the acousto-optical modulator. An optical grid at which the light beam is diffracted is preferably generated in a transparent solid. Particularly preferably, the sound waves responsible for it are generated electrically via the piezo effect. A very rapid electrical influencing of the light beam is thereby possible.

A material which has a junction for the desired wavelength with a basic state that is normally occupied is preferably used as saturable absorber. Integrated in the laser, this material preferably absorbs some of the laser radiation. If the absorber becomes saturated, many states are thus stimulated, the absorption preferably falls, the quality of the resonator exceeds the lasing threshold and laser activity occurs for a short time.

The pulse duration is the period of time in which radiation is produced. This period of time preferably begins with the onset of an increase in radiation and ends when radiation is no longer emitted. The pulse duration is 10 to 10,000 ms, preferably 1 to 10 μs, particularly preferably 1 ms.

The laser preferably has a feature controller to emit a beam in the wavelength range of 500-1064 nm.

A resonator is preferably used as the feature controller, particularly preferably in combination with a temperature regulator or an electrical current. An optical wavelength of the resonator which determines the wavelength of the wave is set by the temperature and/or the current.

In a preferred embodiment, the signal can be generated by the working beam. A signal is thus generated in a simple way. Moreover, the signal is generated directly by the beam which also effects the coagulation.

Here the signal is preferably passed to the detector by reflection from the organic tissue and/or scattering of the working beam. The change in the organic tissue during the coagulation preferably also changes the manner of reflection and/or scattering, particularly preferably at least one property of the reflected and/or scattered beam.

In a further preferred embodiment, the signal can be generated by an auxiliary beam. The signal is thereby preferably generated independently of the working beam. Moreover, if radiation is used as the signal, it is possible to fix the direction of the signal. Furthermore, the properties of the signal can be fixed by the properties of the auxiliary beam. Limitation to the properties of the working beam is not necessary here. Other wavelengths and/or amplitudes and/or frequencies can be used.

An auxiliary beam is a beam which is preferably used exclusively to generate the signal.

Preferably, the auxiliary beam and the working beam can be generated by different sources. It is thereby possible to generate the auxiliary beam independently of the working beam. It is possible to generate the auxiliary beam when there is no working beam or vice versa. Moreover, there are thus particularly great freedoms when fixing the properties of the auxiliary beam. The properties of the auxiliary beam can be fixed in a particularly targeted manner. Thus both the direction of the auxiliary beam and its wavelength or a combination of waves of different wavelengths, amplitude and frequency can be fixed independently.

Any object which emits waves or particles can be used as the source. Light sources, such as lamps, are preferably used, particularly preferably lasers.

The signal is preferably a signal that can be generated by scattering and/or reflection and/or fluorescence excitation and/or thermal excitation. The signal can thereby be generated in a simple way. The signal is generated directly by the changes in the organic tissue.

Scattering is the deflection of the beam caused by interaction with other objects. It is generated here in particular by the lens, the vitreous body and/or the retina. A change in one of these objects preferably produces a change in the scattering. Preferably, a change in the scattering by an alteration on the retina, in particular in the target area of the therapeutic laser radiation is used as the signal.

Reflection is the returning of a wave by a surface. Reflections also occur at the interface of two media with very different wave impedances. The reflection here is preferably diffuse, particularly preferably directed. Where there is a small unevenness of the surface or interface against the wavelength, a directed reflection is achieved, otherwise the reflection is diffuse. Preferably, the signal is here generated by reflection at the retina. A change in the retina or its surface preferably changes the properties of the reflected beam. The direction of the reflection is influenced by a change in the roughness and/or geometry of the surface of the retina. A change in the tissue of the retina preferably changes properties of the reflected beam, such as for example the wavelength or amplitude.

In the case of fluorescence excitation, articles are irradiated with beams of specific wavelengths. This causes the irradiated articles to become excited to a fluorescent radiation. A change in the retina during the coagulation preferably also changes its fluorescent radiation. The degree of coagulation can be determined from the change in the fluorescent radiation. The signal is preferably generated by the exceeding of an intensity or property of the fluorescent radiation.

Light is preferably produced by thermal excitation. Substances emit light when heated. Under certain conditions, solids already display additional light emissions at lower temperatures. This additional radiation occurs only when heating for the first time. Particularly preferably, tissue is changed by thermal influencing. These changes can preferably be recognized visually, particularly preferably they can be ascertained by acoustic effects.

The signal is preferably generated in each case by the exceeding of a limit value for a specific property.

Preferably, the working beam and/or auxiliary beam has waves with a wavelength in the VIS spectral range and/or IR spectral range.

The properties of the working beam or of the auxiliary beam can thereby be controlled in a targeted manner. Particularly preferably, the working beam has waves with a wavelength both in the VIS spectral range and in the IR spectral range. The waves can thereby penetrate deep into the retina and the irradiation of the retina can be carried out in a targeted manner via their depth. Particularly preferably, the auxiliary beam has waves with a wavelength in the VIS spectral range and not in the IR spectral range. This prevents the auxiliary beam from penetrating into the retina and the retina from being unnecessarily heated by waves with wavelengths in the IR spectral range.

The VIS spectral range has waves with wavelengths of 380 to 750 nm. The IR spectral range covers waves with wavelengths of 780 nm to 1 mm.

A laterally and/or vertically defined treatment zone can preferably be scanned by the dimension meter. The condition of the tissue in each area of the treatment zone can thereby be determined. During the scanning, the treatment zone is scanned contact-free, measured values are preferably recorded and particularly preferably stored.

The dimension meter preferably has an OCT detector.

A detector in which temporally short-coherent light is used with the aid of an interferometer, preferably a Michelson interferometer, to measure the distance is preferably used as the OCT detector. The Michelson interferometer preferably has a beam splitter or semi-transparent mirror in which radiation is split up and then re-combined. A photodetector is preferably used, particularly preferably a linear CCD sensor. When measuring the distance, a time- or spectral-domain or frequency-domain process is preferably used.

The dimension meter preferably has a confocal detector.

A detector with a light source, two pinhole diaphragms, a beam splitter and a lens is preferably used as the confocal detector. The excitation light is focused into the sample through one of the pinhole diaphragms, the beam splitter and the lens. This excitation light is preferably reflected by the sample and projected onto a pinhole diaphragm. Preferably, there is an evaluation unit behind the pinhole diaphragm. The light passing through the pinhole diaphragm is preferably evaluated. Preferably, the sample is scanned and an image composed from the results.

The confocal detector is set up in the coagulation system according to the invention to be targeted at a treatment area of the retinal tissue. A detection signal which is recorded by a single photodetector and depends directly on the scattering or absorption at this point can be used according to the invention to assess the progress of the coagulation online. A change in the detection signal can therefore be related directly to the progress of the coagulation. If the change in the detection signal corresponds to a pre-defined value which corresponds to the desired degree of coagulation, the laser exposure of the retinal tissue can be stopped online.

The dimension meter preferably has a confocal OCT detector. It is preferably provided to use a confocal detector and an OCT detector in combination, in order to increase the significance of the signal and thus improve the signal-to-noise ratio. In this confocal OCT arrangement, the change in the detection signal can likewise be assessed and can be used for the online switching-off of the laser exposure of the retinal tissue.

The dimension meter preferably has a laser vibrometer. Acoustic effects which reflect the degree of change of the tissue, for example during laser therapy on the fundus of the eye, are detected using a laser vibrometer.

Laser vibrometers operate on the principle of the Doppler frequency shift. The laser light back-scattered by a vibrating article delivers all the information for the determination of the speed of the object and the absolute oscillation amplitudes.

Various types of laser vibrometers are available to the operator depending on the essence of the task. A scanning vibrometer records the movement of several measurement points at the same time. A single-point vibrometer records the movement of a single measurement point. A 3D laser vibrometer simultaneously records all three directions of acceleration at one measurement point. In a single-pulse irradiation mode, laser vibrometry uses the acoustic signal of a damped oscillation or, in a pulsed multiple-pulse irradiation, the induced tissue oscillations occurring thereby for the analysis.

Preferably, the pilot beam of the therapeutic laser system, which is positioned at the point of the therapy on the fundus of the eye, is simultaneously used for the laser vibrometry in addition to its target function. The described single-point vibrometer is sufficient for this.

In a further embodiment, some of the reflected therapy laser light is used for the laser vibrometry. Particularly preferably, a further independent laser wavelength is used for the laser vibrometry.

The use of this detection technique is particularly advantageous in the non-contact process. It is advantageously used in the case of a fundus camera with an ophthalmoscopic lens as the support system of the therapy laser. Particularly preferably, it is used in the case of a slit lamp with a contact glass.

Preferably the dimension meter has a laser vibrometer in an optically confocal arrangement on the treatment area. Interfering signals which originate from points lying outside the focus area of the therapy spot of the laser are thereby suppressed and the signal-to-noise ratio is improved.

The coagulation system preferably has a localization system for pin-pointing the treatment zone(s) and/or relating the treatment zone(s) to a fundus image. It can thereby be determined at which points an organic tissue has been treated. This is helpful for a later coagulation treatment of organic tissue after an initial treatment.

A coordinate system on which the treatment zones are marked is preferably used as the localization system. The coordinate system is preferably a Cartesian coordinate system, particularly preferably a polar coordinate system.

The picture of a retina is preferably used as the fundus image. Particularly preferably, a picture of the retina of the patient being treated is used.

The localization system according to the invention preferably uses position data of the scanner system which targets the therapeutic laser beam and the associated confocal and/or OCT detector at the respective treatment point on the fundus of the eye. The relationship of these position data to the individual fundus of the eye of the patient is preferably produced by creating the relationship to a retinal coordinate system at the start of the treatment in such a way that significant points, such as the fovea and the papilla, are positioned centrally with the target beam which records the corresponding position data of the scanner unit. Furthermore, associated system parameters are preferably co-recorded, preferably the contact glass used and/or the magnification and/or the picture angle. Correspondingly, the set of treatment data is also available in the case of later interventions and a targeted post-treatment can be carried out and the success of the therapy safely assessed respectively.

Preferably, the localization system according to the invention offers the possibility of intra-operative 2-dimensional marking and/or framing of already treated areas during a follow-up examination and/or post-treatment, e.g. with a correspondingly scanned pilot or target beam.

A scaling for different image-recording modalities is preferably provided in the localization system.

A recording using significant features, preferably the nerve fibre head and/or the macula, particularly preferably the vasculature on the fundus of the eye is preferably provided for the carefully targeted image superimposition.

A calibration of the position data of the laser scanner unit to the respective target image is preferably provided to finally ensure a clear allocation of all the data.

The localization system preferably has a memory for the subsequent pin-pointing of the treatment zones.

For example semiconductor memories, such as flash memories, magnetic memories, such as hard disks, or optical memories, such as CDs, can be used as the memory.

### BRIEF DESCRIPTION OF THE DRAWINGS

The invention will now be illustrated using figures in which further advantageous embodiments are represented. In the figures there are shown:

FIG. 1 a schematic view of a coagulation system according to the invention.

FIG. 2 a schematic view of a second embodiment of a coagulation system according to the invention.

### DETAILED DESCRIPTION

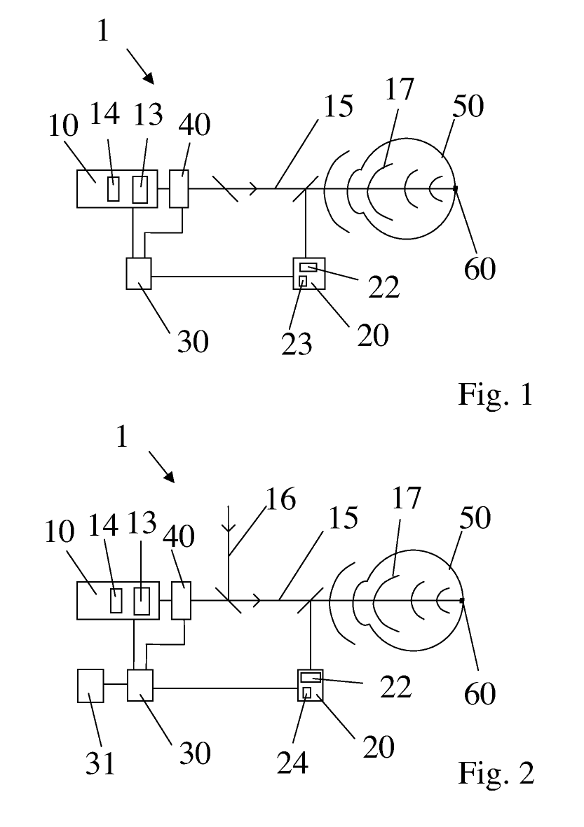

A coagulation system 1 according to the invention is represented in FIG. 1. The coagulation system 1 has a laser 10 with an acousto-optic modulator 13 and a resonator 14, to the output end of which an interrupter 40 is attached. Both the laser 10 and the interrupter 40 are connected to a controller 30. The controller 30 is in turn connected to a detector 20. When the laser 10 is switched on the laser 10 emits a working beam 15. The working beam 15 is targeted at the treatment zone 60 of a retina 50 of a human eye. The working beam is reflected by the retina 50 as a spherical wave 17. A part of the spherical wave 17 is redirected to the detector 20. In a confocal arrangement of the detector 20 which is targeted at the treatment zone 60, the indirectly back-scattered laser light or fluorescent light is detected with a diaphragm ring.

The laser 10 alternatively emits radiation with one wavelength or with up to four different wavelengths. The acousto-optic modulator 13 switches very quickly between blocking and letting through, with the result that the waves are pulsed. The resonator 14 is modular in structure and can optionally bring about the emission of four different wavelengths (e.g. yellow, green, red, infrared).

This laser radiation brings about a coagulation effect on the retina. The blood vessels of the retina inter alia are thereby closed here. The retina is divided into different areas to which different therapy zones are assigned. Different irradiation profiles, i.e. sequences of irradiation with waves having specific wavelengths in the VIS and/or IR range, are applied in the different therapy zones. The power output of the source is pulsed to carry out selective retina therapy (SRT). The pulse duration in which power is emitted is 5 μs. This short-pulse 5 μs radiation can be applied with a repetition frequency of 100 Hz (10-10,000 Hz). The principle of the selective thermolysis already very largely spares the photoreceptor layer and only the retinal pigment epithelium (RPE) is coagulated locally. According to the invention, an individually adapted pulse burst length is generated in this operating regime using the detector 20. Thus, in the case of pre-defined change in the detection signal to a limit value, the pulse burst which is quasi-continuous with e.g. 100 Hz/5 μs is interrupted using the interrupter 40 or else the acousto-optic modulator 13.

The light energy is absorbed by the tissue of the retina, converted into heat and thereby leads to a denaturation or coagulation of the tissue. The retina is discoloured by a complete coagulation. The area of the retina in which there is a complete coagulation no longer supports vision. If the area of the retina at which the laser is targeted displays a coagulation, the reflected spherical wave 17 changes. This changed spherical wave 17 is redirected to a detector 20. The detector 20 has an OCT detector 23 which irradiates its measurement beam coaxially to the therapy laser 10 onto the treatment zone 60 and at the same time superimposes the back-scattered signal with an internal reference signal position-accurately in respect of the treatment zone. If a previously fixed change in the OCT signal is reached, the detector 20 sends this information to the controller 30. The controller 30 then switches the laser 10 off.

The laser 10 is switched off online when the very first changes occur in the tissue, in order to avoid collateral tissue damage, in particular of the photoreceptor layer. In order to take account of the different treatment requirements and the different optical and thermal properties of the different tissue layers of the retina, the coagulation system is set up to be able to interrupt different treatment wavelengths in the visual and infrared range independently of each other. The effect on the tissue can therefore be controlled both temporally and spectrally. Waves with wavelengths in the infrared range are interrupted here when a sufficient coagulation has taken place in deeper tissue layers of the retina.

Damage to the surrounding tissue can be very largely prevented with this coagulation system.

FIG. 2 shows a second embodiment of a coagulation system according to the invention. In FIG. 2, the signal is generated by an auxiliary beam 16, unlike the coagulation system represented in FIG. 1. The auxiliary beam 16 here has a wavelength in the visual spectral range. It is conducted onto the retina 50, from there reflected as a spherical wave and conducted onto a detector 20. The detector 20 has a confocal detector 24 by which the wavelength of the beam can be determined. The area of the retina 50 on which the auxiliary beam 16 lands is changed by a change in the direction of the auxiliary beam 16. Thus, the detector 20 can examine the condition of the retina in different areas.

The measurement point of this OCT detector 23 is targeted at the “coagulation” spot of the laser or therapy laser 10. A detection signal which is recorded by a single photodetector or a linear CCD sensor and depends directly on the scattering or absorption at this point is used according to the invention in order to assess the progress of the coagulation online. The change in the detection signal can therefore be directly related to the progress of the coagulation. If the alteration of the detection signal corresponds to a pre-defined value which corresponds to the desired degree of coagulation, the laser exposure of the retinal tissue is stopped online.

A further difference from the coagulation system represented in FIG. 1 is that the controller 30 is connected to a recording system 31. This recording system records the position and intensity of the treatment. It is set up to display the treatment positions on a fundus image taken previously. A subsequent pin-pointing of the treatment zones is thereby made possible. This is helpful for follow-up treatments.

### LIST OF REFERENCE NUMBERS

1 coagulation system

10 laser

11 continuously emitting source

12 clock generator

13 acousto-optic modulator

14 resonator

15 working beam

16 auxiliary beam

17 spherical beam

20 detector

22 dimension meter

23 OCT detector

24 confocal detector

30 controller

31 recording system

40 interrupter

41 wavelength interrupter

50 retina

60 treatment zone

70 localization system

71 memory

1-18. (canceled)

19. A coagulation system for the coagulation of organic tissue comprising:
a laser configured to emit a working beam;
an interrupter configured to at least partially interrupt the working beam;
a controller configured to activate the interrupter; and
a detector configured to detect a signal corresponding to a degree of coagulation or alteration of the tissue and to transmit a detection of the signal to the controller, the detector including a dimension meter.

20. The coagulation system as recited in claim 19, wherein the organic tissue includes a retina.

21. The coagulation system as recited in claim 19, wherein the laser includes a clock generator configured to pulse the working beam between 10 ms and 10,000 ms.

22. The coagulation system as recited in claim 19, wherein the laser includes a pulse generator configured to pulse the working beam with a pulse duration of between 2 and 10 μs.

23. The coagulation system as recited in claim 22, wherein the pulse duration is between 3 and 7 μs.

24. The coagulation system as recited in claim 22, wherein the pulse duration is 5 μs.

25. The coagulation system as recited in claim 19, wherein the laser includes a property controller configured to emit radiation having a wavelength between 500 and 1064 nm.

26. The coagulation system as recited in claim 19, wherein the interrupter includes a wavelength interrupter configured to interrupt waves of the working beam having specific wavelengths.

27. The coagulation system as recited in claim 26, wherein the wavelength interrupter is configured to interrupt waves of the working beam having wavelengths in the infrared range.

28. The coagulation system as recited in claim 26, wherein the wavelength interrupter is configured to interrupt waves independently of a detected coagulation.

29. The coagulation system as recited in claim 19, wherein the working beam is configured to generate the signal.

30. The coagulation system as recited in claim 19, wherein an auxiliary beam is configured to generate the signal.

31. The coagulation system as recited in claim 30, wherein the auxiliary beam and the working beam are generated by different sources.

32. The coagulation system as recited in claim 19, wherein the signal is generated by at least one of scattering, reflection, fluorescence excitation and thermal excitation.

33. The coagulation system as recited in claim 30, wherein at least one of the working beam and the auxiliary beam include a wavelength in at least one of a visible spectral range and an infrared spectral range.

34. The coagulation system as recited in claim 19, wherein the dimension meter is configured to scan at least one of a laterally and a vertically defined treatment zone.

35. The coagulation system as recited in claim 19, wherein the dimension meter includes an optical coherence tomography (OCT) detector.

36. The coagulation system as recited in claim 19, wherein the dimension meter includes a confocal detector.

37. The coagulation system as recited in claim 19, wherein the dimension meter includes a confocal OCT detector.

38. The coagulation system as recited in claim 19, wherein the dimension meter includes a laser vibrometer.

39. The coagulation system as recited in claim 38, wherein the laser vibrometer is disposed in an optically confocal arrangement on the treatment area.

40. The coagulation system as recited in claim 19, further comprising a localization system configured to at least one of pin-point a treatment zone and relate the treatment zone to a fundus image.

41. The coagulation system as recited in claim 40, wherein the localization system includes a memory configured to subsequently pin-point the treatment zone.
